(19) United States
(12) Patent Application Publication
(10) Pub. No.: US 2002/0136777 A1
(43) Pub. Date: Sep. 26, 2002

(54) Functional antidiabetic drink

(76) Inventors: Jin-Ho Choi, Pusan (KR); Kang-Sun Ryu, Ansan-si (KR); Heui-Sam Lee, Kyonggi-do (KR); Dong-Woo Kim, Pusan (KR); Dae-Ik Kim, Kyongsang-pukto (KR); Soo-Hyun Park, Pusan (KR)

Correspondence Address: PERKINS, SMITH & COHEN LLP, ONE BEACON STREET, 30TH FLOOR, BOSTON, MA, 02108, US

(21) Appl. No.: 09/747,811

(22) Filed: Dec. 22, 2000

(51) Int. Cl. A61K 35/24; A61K 35/12; A61K 35/37; A61K 35/64

(52) U.S. Cl. 424/520; 424/537

(57) ABSTRACT

Diabetic inhibiting drink with an extract of silkworm protein.

### FIELD OF THE INVENTION

The present invention relates to a functional antidiabetic drnks which is intended to prevent the diabetic disease.

### BACKGROUND OF THE INVENTION

According to the WHO report of 1985, diabetes is classified into: an insulin dependent diabetes (type I), an insulin non-dependent diabetes (type II), and a malnutritional diabetes (MRDM). Fortunately, about 84%. of the Korean patients belong to the type II (the insulin non-dependent diabetes) (reported by Lee et al, in 1984). Paticularly, in the adults of 40 years old or over, the type II diabetes occurs frequently in the following persons (1) those who have a high obesity, (2) those who are low in activities, (3) those who have hypertensive syndromes, (4) those who have an abnormality in their livers, and (5) those who have a high content of neutral lipid.

In view of the above facts, the present inventor focused on the fact that the above five causes for the diabetes are same as the cause of the chronic degenerative disease of the present days, and thus, the present inventor came to invent a functional antidiabetic drink whicd is capable of preventing the disease and the related syndromes. About 84% of the Korean diabetic patients are the insulin non-dependent type patients as described above, and therefore, a natural substance rather than insulin will be more beneficent, thereby producing a demand for such a natural substance. Accordinaly the silkworm powders which has been the folk therapy is prepared into a silkworm extract, and this is made into a functional antidiabetic drink which is a physiologically active drink.

### SUMMARY OF THE INVENTION

So far, the research on the diabetes inhibiting effect has been carried out in such a manner that the silkworm powders are administered into an arnmal to carry out a clinical experiment.

It is an object of the present invention to provide a functional antidiabetic drink in which three days old silkworms are freeze-dried (to prevent the destruction of the ingredients) and are powdered, then an extraction is carried out by using methanol, then a low pressure condensing is carried out, and then a freeze-drying is carried out, thereby obtaning a silkworm extract without destruction of the ingredients. If the silkworm passes several days after the optimum age of three days, the therapeutic effect is drastically decreased.

About 84% of the Korean diabetic patients are the insulin non-dependent type patients (type II), and therefore, it was confirmed that a natural substance having a therapeutic efficacy is needed rather than insulin to prevent diabetes, and therefore, and that such a drink is needed. Therefore. a functional antidiabetic drink came to be developed. That is, a daily dosage of 30 mg of a mulberry leaf extract (MLE), a silkworm extract (SWE) and a silk fibroin powder (SFP) was peritoneally administered on SD rats to whom diabetes was induced for four days by using streptozotocin. The extracts were administered after diluting them in an amount of water of 0.5 ml, and this antidiabetic drug therapy was evaluated after 12 days.

After the evaluation, the silkworm extract (SWE) which showed the most superior efficacy was administered on the SD rats (in whom diabetes was induced by injecting 60 mg/kg BW of streptozotocin for four days) by varying the dosage to 10 mg, 30 mg. and 60 mg. Meanwhile, as comparative groups, the antidiabetic drug Daonil (Handock Pharmaceuticals) was administered on the rats by varying the dosage at 40 mg (1.25 mg of glybenclamide) and 80 mg (2.50 mg of glybenclamide). Through this experiment, it was confirmed that the silkworm extact was as much efficacious as Daunil of Handoclk Pharmaceuticals. Accordingly, the functional antidiabetic drink of the present invention came to be developed.

### DETAILED DESCRIPTION OF THE PREFERRED EMBODIMENTS

The animal experiments, the extraction of the silkworm powders, the experiment materials, the experiment method and the experiment results will be described below.

1. Animal Experiments and Feeding Stuff

(1) Expeimented Animals and Experimentation

SD rats (female, 160+−10 g) were procured from Korean

Chemical Laboratory, and a pre-raising was carried out for two weeks. Then diabetes was induced in than by using streptozotocin (STZ), and on the fourth day, the rats who showed a glucose level of 300 mg/dl or more were selected to use them in the experiment. In an animal experiment I, the experiment group was divided into 7 groups, with each group including 7 rats. A control group was peritoneally administered with water. In order to check the antidiabetic drug efficacy, the mulberry (morus alba) leaf extract (MAE), the silkworm (mobyx mori. L.) extract (SWE) and the silk fibroin (SF) were respectively administered at a daily dosage of 30 mg.

In an animal experiment II, the experiment group was divided into 7 groups, with each group including 7 rats. The control group was peritoneally administered with water. The silkworm extract (SWE) which showed the most superior efficacy was admninistered on the SD rats (in whom diabetes was induced by injecting 60 mg/kg BW of streptozotocin for four days) by varying the dosage at 10 mg, 30 mg, and 60 mg. Meanwhile, as a comnparative group the antidiabetic drug Daonil (Handock Pharmaceuticals) was administered on the same rats by varying the dosae at 40 mg (1.25 mg of glybenclamide) and 80 mg (2.50 mg of glybenclamide, this dosage corresponding to the daily dosage for a human adult). The drug was administered after diluting it in an amount of water of 0.5 mg, and the adninistration was continued for 12 days. Then the glucose lowering effect was analyzed, thereby evaluating the drug efficacy,

The animal feeding was carried out at a constant temperature and humidity (22+-2 degrees C., 65+20% RH), and the illumination and darkness were continued for 12 hours alternately repeatedly (06:00 - 18:00).

(2) Preparation of The Feeding Stuff

The feeding stuff included: 59.0% of carbohydrate (alpha-corn starch: 44.0% +sucrose: 15.0%); 18.0% of protein (sodium-fee casein), 15.0% of lipid (lard; 10.0% +corn oil: 5.0%); 1.0% and 3.5% of vitamin and inorganic materials (AIN-76 mixture); 3.0 of fibrous materials; 0.3% of DL-methionine, and 0.2% of choline chloride.

2. Experimented Materials

(1) Materials for Developing The Antidiabetic Drink

The mulberry leaf extract, the silkworm extract and the silk fibroin were procured from the Agricultural Promotion and Agricultural Science and Research Center to use them in the experiments on the antidiabetic efficacy. In order to comparatively evaluate them, Daonil (which is the antidiabetic drug for the insulin non-dependent diabetes) of Handock Pharmaceuticals was procured to use it in the comparative experiment.

(2) Reagents Used

The reagents used in the experiment of the present invention included: streptozotocin for inducing diabetes: a kit reagent for measuring the glucose (which is the blood sugar); and other roagents fo the anaysis and manufactured by Sigma company.

3. Expexmental Method

(1) Inducing The Diabetes

Strepozotocin (STZ) was administered on the SD rats in a dosage of 60 mg/kg BW by diluting it in a 0.1 M sodium citrate buffer (pH 4.3), the injection being made through the tail vein. The blood was drawn from the tail vein at 0th, 2nd and fourth days to analyze the level of glucose. Then the rats. who showed a glucose level of 300 mg/dl or more on the fourth day were selected for the expement. Then in the animal experiment I, the mulbery leaf extract (MAE), the ilkworm extract (SWE) and the silk fibroin (SF) were peritoneally administered into the seclected rats for 12 days, and during this period, the glucose levels were compared and evaluated. In the animal experiment II, the silkwornm extract (SWE) and Daonil which was commercially available were peitoneally administered for 12 days to analyze it so as to carry out a comparative evaluation.

(2) Measurement of Blood Glucose

In the blood drawn from the tail vein of the rats, the glucose level in the blood serum was measured by using the kit reagent (Sigma company of USA) which was based on the enzyme method). First, blood serum and glucose standard flud (400 mg/dl) were added in an amount of 5 micro L respectively, then a coloring reagent was added in an amount of 1.0 ml, and then, a mixing was carried out. Thus, a reaction was carried out at a temperature of 37 degrees C. for 15 minutes. A blank into which a distilled water and the coloring reagent were put was adopted; and the beam absorbability was measured at a wavelength of 505 nm. Then the glucose level was calculated based on the following formula:

Glucose level (mg/dl senum)=(OD material to be checked/OD standard solution)×400* where * indicates the concentration of the glucose standard solution.

(3) Measurement of Lipid and lipid-peroxide

One of the causes of the insulin non-dependent diabetes (type II) is the accumulation of the neutral lipid. Accordingly, the triglyceride which is the neutral lipid of the blood serum was measured by the kit reagent (Sigma company of USA). Further, This neutral lipid is known to be formed by being intruded by an active oxygen. The lipid peroxide (LPO) which is formed by being intruded into the lipid of the blood serum was measured as to its beam absorbability at a wavelength of 535 nm by a spectro-photometer based on the conventional method (1991).

(4) Measurement of Active Oxygen and The Removing Enzyme

Hydroxyl radical (OH) which is known to be the most strong active oxygen which forms an aldehyde through the decomposition of deoxyribose by the reactive oxygen product. Based on this fact, the phenomenon that the aldehyde formed during the reaction is reacted with thiobabituric acid within the acidic solution to be colorized, and thus, the measurement was carried out bascd on the mcthod of Halliwell et al. (1981). The measurement of superoxide dismutase (SOD) as the bio defense system was carried out based on the method of Oyanagi et al. (1984).

4.Evaluation of Experimental Results

A. Result of The Animal Experiment I

(1) Glucose Lowing Effect of The Silkworm Related Products

In the animal experiment I, the efficacy of the silkworm related products such as mulberry leaf extract, the silk fibroin and the silkworm extract were analyzed and compared to each other as to their glucose level lowering effects. In the rats, diabetes was induced by injecting streptozotocin (STZ) for four days, and then, the rats who showed 300 mg/dl or more were divided into four groups, each group including 7 rats. In a control groups, water was peritoneally administered. Then, in order to elicit the antidiabetic effect, the mulberry leaf extract (MLE), the silkworm extract (SWE and the silk fibroin powder were administered at a dosage of 30 mg/kg BW for 12 days. Then the glucose inhibiting effects wer mcasured for the respective groups, and the measured results are shown in FIG, 1 below.

[image]

As shown in FIG. 1 above. the mulbmy leaf extract (MLE) could not expect any significant glucose lowering effect. However, the silk fibroin powder (SFP) and the silkworm extract (SWE) began to show the glucose lowerng effect from the second day after the administration. Starting from the fourth day after the administration, a glucose lowering effects of 10% and 20% were seen compared with the control group. The silk fibroin powder (SFP) showed an effect of about 20% from 6th day to 12th day, while the silkworm extract (SWE) showed a more significant effect of 20 - 3096 from the fourth day to the 12th day.

B. Result of Animal Experiment-II

(1) Comparison With The Antidiabetic Drug for The Glucose Lowering Effect

In the animal experiment I, the silkworm extract SWE showed a far greater glucose lowering effect. That is, compared with the control group, a glucose lowering effect of about 20 -30% was seen. Accordingly, diabetes was induced by using streptozotocin for four days, and then, the rats who showed a blood sugar level of 300 mg/dl or more were selected and were divided into 6 groups, each group incluing 7 rats. A control group was administered with water. In order to elicit the glucose lowering effect, the silkworm extract (SWE) was administred in amounts of 10, 30 and 60 mg, and Daonil (glybenclamide) of Handock Pharmaceuticles (which contracted a technical assistance agreement with the Hoechst Pharmaceuticals) was administered in amounts of 40 mg (glybenclamide: 1.25 mg) and 80 mg (glybenclamlide: 2.50 mg) after diluting them in water of 0.5 ml. The administrations were carried out through the bellies for 12 days, and the glucose lowering effects were compared to cach other. Table I below chows the compared results.

As shown in Table 1 below, the SWE-10 group showed a significant glucose lowering effect starting from the second day after the administration in contrast to the control group, until the finer group shuwed a glucose loweing effect of about 20% on the 12th day. The SWE-30 group showed a significant glucose lowering effect of about 15% startng from the second day after the administration compared with the control group, until the former group showed a glucose lowing effect of about 25% on the 12th day. The SWE-60 group showed a significant glucose lowering effect of more than 15% starting from the second day after the administration compared with the control group, until the former group showed a glucose lowering effect of more than 30% on the 12th day,

TABLE 1
Influence of silkworm extract (SWE) and Daonil on the
glucose lowering effect

| | | | | | Daonil administered | |
|---|---|---|---|---|---|---|
| Period(days) | | Silkworm extract administered group | | | group | |
| Administered | Control Group | SWE-10 | SWE-30 | SWE-60 | Daonil-40 | Daonil-80 |
| 0(7)* | 548.7 ± 41.2** | 512.7 ± 43.5 | 514.5 ± 45.2 | 518.6 ± 38.6 | 536.0 ± 42.6 | 541.9 ± 40.9 |
| | 100.0% | 93.4% | 93.8% | 94.5% | 97.7% | 98.8% |
| 2(7) | 560.5 ± 45.9 | 484.8 ± 35.6^{c} | 468.6 ± 42.5^{b} | 461.1 ± 40.6^{b} | 461.2 ± 38.4^{b} | 398.3 ± 34.5^{c} |
| | 100.0% | 88.1% | 85.1% | 83.8% | 83.8% | 72.4% |
| 4(7) | 473.8 ± 39.7 | 407.7 ± 37.5^{b} | 391.4 ± 43.4^{c} | 392.4 ± 39.6^{c} | 386.5 ± 33.3^{c} | 372.7 ± 31.2^{c} |
| | 100.0% | 86.1% | 82.6% | 82.8% | 81.6% | 78.7% |
| 6(7) | 475.5 ± 51.3 | 394.2 ± 29.8^{b} | 371.4 ± 29.7^{c} | 372.6 ± 35.5^{c} | 370.8 ± 27.5^{c} | 368.5 ± 30.0^{c} |
| | 100.0% | 82.9% | 78.1% | 78.4% | 78.0% | 77.5% |
| 8(7) | 475.7 ± 48.6 | 395.4 ± 32.9^{b} | 363.6 ± 34.7^{c} | 354.4 ± 28.5^{c} | 344.4 ± 26.5^{c} | 363.6 ± 38.3^{c} |
| | 100.0% | 83.1% | 76.4% | 74.5% | 72.4% | 76.4% |
| 10(7) | 462.6 ± 35.8 | 390.7 ± 33.7^{b} | 360.6 ± 35.2^{c} | 349.9 ± 32.1^{c} | 347.6 ± 29.2^{c} | 351.4 ± 28.4^{c} |
| | 100.0% | 84.5% | 78.0% | 75.6% | 75.1% | 76.0% |
| 12(7) | 450.2 ± 43.5 | 352.4 ± 35.3^{c} | 325.6 ± 28.8^{c} | 311.4 ± 25.4^{c} | 286.5 ± 25.5^{c} | 293.5 ± 25.6^{c} |
| | 100.0% | 78.3% | 72.3% | 69.2% | 63.6% | 65.2% |

*number of animals emplored :
**average value ± SD :
***SWE-10, 30, 60 : Groups administered with daily dosages of 10, 30, 60 mg :
****Daonil-40, 80 : Groups administered with antidiabetic drug Daonil in dosages of 40, 80 mg daily :
significance checked : in comparison with the control group
^{a}p < 0.05 :
^{b}p < 0,01 :
^{c}p < 0.001

Further, in order to evaluate the glucose lowering effect of the silkworm extract, the commercially available Daonil the product of Handock Pharmaceuticals was administered. The Daonil-40 group showed a significant glucose lowering effect of 15% or more starting from the second day after the administration, and on the 12th day, a more significant effect of 35% or more was seen. The Daonil-80 group showed a significant glucose lowering effect of about 25% starting from the second day after the administration, and on the 12th day, a more significant effect of 35% or more was seen like the Daonil-40 group.

As can be seen in the above experiments, it was proved that the silkworm extract (SWE) administered groups showed a significant glucose lowering effect comparable to the Daonil administered groups. In view of the fact that the s13kworm extract gives a wonderful antiglucose effect comparible to the Daonil the antidiabetic drug, and in view of the fact that the silkworm extract gives no adverse effect unlike Daonil, it can be assessed that the silkworm extract is a desirable thing for the diabetic patients,

(2) Lipid and Lipid-Peroxide Inhibiting Effect

The obesity and the accumulation of the neutral lipid can become the cause of the insulin non-dependent diabetes (type II). Therefore, for the rats which showed a glucose level of 300 mg/dl, the control group was peritoneally administered with water, while the experimental groups were administered with the silkworm extract (SWE-10, 30 and 60 groups), and were administered with Daonil the antidiabetic drug (Daonil-40 and 80 groups). In these groups, the neutral lipid and lipid-peroxide inhibiting effects were compared to each other, and the comparison results are shown in Table 2 below. As shown, in this table, the SWE,-30 group showed a significant neutral lipid inhibiting effect, and the SWE-60 group showed a more significant effect of 15% or more. Meanwhile, the Daonil-40 group showed a neutral lipid inhibiting effect of about 13%, and the Daonil-80 group showed a neutral lipid inhibiting effect of about 30%, Accordingly it can be construed that the antiglucose effect of the silkworm extract and Daonil woos to the inhibition of the neutral lipid.

Meanwhile, as can also be seen in Table 2, the SWE-30 group and the SWE-60 group showed about 10% and 15% of a lipid peroxide inhibiting effects respectively, while the Daonol-40 group and the Daonil-80 group showed a lipid peroxide inhibiting effect of about 15%. Therefore, it can be construed that the silkworm extract inhibits the lipid peroxide like Daonil the antidiabetic drug to cure diabetes.

TABLE 2
Influence of silkworm extract and Daonil on lipid and
lipid-peroxide after 12 days

| | | silkworm extract administered | | | Daonil administered | |
|---|---|---|---|---|---|---|
| | control | group** | | | group*** | |
| Classification | group | SWE-10 | SWE-30 | SWE-60 | Daonil-40 | Daonil-80 |
| neutral lipid | 89.13 ± 8.00* | 86.81 ± 6.23 | 80.62 ± 7.54^{c} | 74.80 ± 3.40^{b} | 77.44 ± 6.49^{b} | 62.20 ± 4.39^{c} |
| | 100.0% | 97.4% | 90.4% | 83.9% | 86.9% | 69.8% |
| lipid peroxide | 0.38 ± 0.01 | 0.40 ± 0.02 | 0.35 ± 0.02^{a} | 0.33 ± 0.04^{a} | 0.32 ± 0.02^{b} | 0.32 ± 0.03^{b} |
| | 100.0% | 105.3% | 92.1% | 86.8% | 84.1% | 84.1% |

*average value ± SD
**SWE-10, 30, 60 : groups administered with daily dosages of 10, 30, 60 mg of silkworm extract
***Dionil-40, 80 : groups administered with daily dosages of 40, 80 mg of antidiabetic drug Daonil :
significance checked : in comparison with control group :
^{a}p < 0.05 :
^{b}p < 0,01 :
^{c}p < 0.001

(2) Evaluation of Activities of Active Oxygen and Removing Enzyme

Up to the present, attention has been focused on the oxidative theory among the serility theories. The influences of the silkworm extract and the antidiabetic (Daonil) on the active oxygen and the removing enzyme which affect the insulin non-dependent diabetes (type II) were compared and analyzed, and the results are shown in Table 3 below.

As shown in Table 3, the hydroxyl radical (.OH) which is most toxic to the mature adult diseases and to the senility among the kinds or active oxygen was analyzed. The SWE-10 group showed a significant .OH radical inhibiting effect of about 15%. The SWE-30 and −40 groups showed a significant .OH radical inhibiting effect of about 20%. Meanwhile the Daoni-40 and -80 groups showed a OH radical inhibiting effect of about 7-12%. Accordingly, it was proved that the silkworm extract is more efficacious in inhibiting the active oxygen than Daonil the antidiabetic drug.

As to the activity of superoxide dismutase (SOD) which is the most izmpftant among the bio defense enzymes, the SWE-10, SWE-30 and SWE-60 groups showed a significant SOD activity increasing effect of about 10-15%, while the Daonil-40 and Daonil-80 groups showed an SOD activity increasing effect of about 20-30%. Thus the silkworm extract also gives a considerable SOD activity increasing effect like Daonil the antidiabetic drug.

TABLE 3
Influence of silkworm extract and Daonil on increasing
the activity of active oxygen and the removing enzyme after 12 days

| | | silkworm extract administered | | | Daonil administered | |
|---|---|---|---|---|---|---|
| | control | group*** | | | group**** | |
| Classification | group | SWE-10 | SWE-30 | SWE-60 | Daonil-40 | Daonil-80 |
| Active | 3.82 ± 0.48** | 3.28 ± 0.16^{b} | 3.10 ± 0.15^{c} | 3.08 ± 0.30^{c} | 3.54 ± 0.31^{a} | 3.35 ± 0.30^{a} |
| oxygen | | | | | | |
| (.OH) | 100.0% | 85.9% | 81.2% | 80.6% | 92.7% | 87.7% |
| SOD | 0.35 ± 0.02 | 0.38 ± 0.03^{a} | 0.39 ± 0.04^{a} | 0.40 ± 0.04^{b} | 0.42 ± 0.03^{c} | 0.45 ± 0.04^{c} |
| enzyme* | | | | | | |
| | 100.0% | 108.6% | 111.0% | 114.3% | 120.0% | 128.6% |

*SOD : superoxide dismutase
**average value ± SD :
***SWE-10, 30, 60 : groups administered with silkworm extract in daily dosages of 10, 30, 60 mg
****Daonil-40, 80 : groups administered with antidiabetic drug Daonil in daily dosages of 40, 80 mg :
significance checked in comparison with control group :
^{a}p < 0.05 :
^{b}p < 0,01 :
^{c}p < 0.001

To summarize the results of the research, the silkworm extract was most efficacious in the antidiabetic effect among the silkworm related products such as mulberry lcaves, the silk fibroin and the silkworm. Further, the silkworm extract was comparable in its antidiabetic effect to the antidiabetic drug Daonil. Futher, the silkworm extract was efficacious to the same degree in inhibiting the neutral lipid, the active oxygen, and the lipid peroxide, and in increasing the activities of the SOD which is the bio defense enzyme. In view of the experimental results. the antidiabetic effect can be sufficientiy obtained with a daily dosage of 30 to 60 mg, but in over to prevent the diabetes, a functional antidiabetic drink containing a weight part of 0.1 is provided in which the physiologically activating ingredients are preserved.

<Example 1>

The functional antidiabetic drink was manufactured in the following manner. That is, the method for manufacturing the functional antidiabctic drink according to the present invention includes the steps of: freeze-drying silkworms having an age of 3 days; carrying out an extraction by using methanol and carrying out a low pressure condensing; adding 0.05 weight parts of ginger extract and 0.05 weight parts of cinnamon bark extract for each 0.1 weight tarts of the silkworm extract (in which freeze-dried plhysiologically activating ingredients are preserved) so as to eliminate displeasing odors; adding 0.05 weight parts of vitamin C and 0.05 weight parts of citric acid to promote taste and flavor; adding 0.1 weight parts of taurine as an anti-exhaustion agent; carrying out a mixing; carrying out a centrifugal separation at 1000×g for 10 minutes; and taking an upper fluid by an amount of 100 ml, Thus the antidiabetic drink was manufactured. That is, the compositions were varied as shown in Table 4 below, and the comparative experiments were carried out. The result showed that the above described composition was most effective.

TABLE 4
Contents of the ingredients of the functional antidiabetic drink.

| Ingredient/ | | | | | | | | | | | | |
|---|---|---|---|---|---|---|---|---|---|---|---|---|
| volume | 0.001 | 0.010 | 0.050 | 0.100 | 0.25 | 0.50 | 1.00 | 2.50 | 5.00 | 10.0 | 15.0 | 20.0 |
| silkworm | | ● | | ● | | | ● | | | | ● | |
| extract | | | | | | | | | | | | |
| ginger | ● | | ● | ● | | | ● | | | | | |
| extract | | | | | | | | | | | | |
| cinnamon | ● | | ● | | | ● | | ● | | | | |
| bark extract | | | | | | | | | | | | |
| Taurine | | ● | | | ● | | ● | | | ● | | |
| citric | ● | ● | | | ● | | ● | | | | | |
| acid | | | | | | | | | | | | |
| vitamin C | ● | ● | | | ● | | ● | | | | | |

Note: actually prescribed ingredients and their volume added

Now the effects of the present invention will be descrbed.

The insulin non-dependent diabetic patients (type II) constitute 84% of the whole diabetic patients, and this disease is caused by the meat preference and the consequent ingestion of the neutral lipid and the insufficiency of the athletic exercise, which are the form of the recent trend of the society. Other this disease is most diflicult to be cured. Accordingly in the present invention, the silkworm related products such as mulberry leaf extract, the silk fibroin powder and the silkworm extract were expented to the diabetic disease. The mulberry leaf extract showed no significant efficacy, but the silk fibroin powder (SFP) gave a glucose lowering effect of about 20% on the 12th day after the administration, while the silkworn extract (SWE) gave a significant glucose lowering effect of 30% on the 12th day after the administration.

Meanwhile, Daonil the antidiabetic drug of Handock Pharmaceuticals was administered and the results were analyzed and evaluated. The result showed that the SWF-30 and SWE-60 groups showed a significant glucose lowering effect of 25% and 30% respectively on the 12th day afer the administration compared with the control group, while the Daonil-40 and 80 groups showed a glucose lowering effect of 35% on the 12th day after the administration compared with the control group. Thus the silkworm extract showed an effect altnost comparable to Daonil the antidiabetic drug.

Further, the silkworm extract and Daonil of Handock Pharmaceuticals were experimented for their effects on inhibiting the neutral lipid, the lipid peroxide and the active oxygen, and on the activity of the SOD. The experimental results were as follows. That is, the SWE-10, 30 and 60 groups showed the inhibiting effects of 10-15%, 10-15% and 15-209% rcspectively on the 12th day after the administration compared with the control group, while the Daonil-40 and 80 groups showed an inhibiting effect of 15-20%, 159, and 5-12% respectively on the 12th -day after the administration compared with the control group. The activity of the SOD (superoxide dismutase) as the bio defense enzyme was as follows. The SWP-10, 30 and 60 groups showed the increasing effects of 10-15% respectively on the 12th day after the administration compared with the control group, while the Daonil-40 and 80 groups showed an increasing effect of 20 - 30% on the 12th day af the administration compared with the control group. In view of the above described results, the functional antidiabetic drink according to the present invention should be efficacious in preventing the diabetic disease.

What is claimed is:

1. A functional antidiabetic drink comprising:
a silkworm extract extracted from silkworms of an age of 3 days by using methanol, and low-pressure-condensed;
0.05 weight parts of ginger extract and 0.05 weight parts of cinnamon bark extract for each 0.1 weight parts of the silkworm extract (in which freeze-dried physiologically activating ingredients are preserved), for eliminating displeasing odors;
0.05 weight parts of vitamin C and 0.05 weight parts of citric acid, for promoting taste and flavor: and
0.1 weight parts of teurine as an anti-exhaustion agent.

2. A method for manufacturing a functional antidiabetic drink, cnmprisinng the steps of:
freeze-drying silkworms having an age of 3 days;
carrying out an extraction by using methanol, and carrying out a low pressure condensing;
adding 0.05 weight parts of ginger extract and 0.05 weight parts of cinnamon bark extract for each 0.1 weight parts of the silkworm extract (in which freeze-dried physiologically activating ingredients are preserved) so as to eliminate displeasing odors;
adding 0.05 weight parts of vitamin C and 0.05 weight parts of citric acid to promote taste and flavor;
adding 0.1 weight parts of tawne as an anti-exhaustion agent;
carrying out a mixing;
carryihg out a centrifugal separation at 1000×g for 10 minutes; and
taking an upper fluid by an amount of 100 ml.
